(19) United States
(12) Patent Application Publication
(10) Pub. No.: US 2018/0174320 A1
(43) Pub. Date: Jun. 21, 2018

(54) Posture Detection Device and Posture Detection Method

(71) Applicant: Konica Minolta, Inc., Tokyo (JP)

(72) Inventors: Shuji Hayashi, Hino-shi, Tokyo (JP); Koji Fujiwara, Shimamoto-cho, Mishima-gun, Osaka (JP)

(73) Assignee: Konica Minolta, Inc., Tokyo (JP)

(21) Appl. No.: 15/555,869

(22) PCT Filed: Mar. 2, 2016
(86) PCT No.: PCT/JP2016/056496

§ 371 Date: Jan. 8, 2018

(30) Foreign Application Priority Data
Mar. 6, 2015 (JP) 2015-044627

(51) Int. Cl. G06T 7/73; G06K 9/00; A61B 5/11

(52) U.S. Cl. CPC G06T 7/73; G06K 9/00335; G08B 21/043; A61B 5/1128; A61B 5/1117

(57) ABSTRACT

In a posture detection device and a posture detection method of the present invention, an image of a prescribed detection area is acquired by an image acquisition unit, a head portion is extracted from the acquired image of the detection area, a prescribed parameter for the extracted head portion is determined, and whether a prescribed posture is taken is determined on the basis of the determined parameter. Therefore, in the posture detection device and posture detection method of the present invention, the posture of a monitored subject can be determined more precisely with a simpler configuration.

### TECHNICAL FIELD

The present invention relates to a posture detection device that detects the posture of a monitored subject and to a posture detection method.

### BACKGROUND ART

Our country (Japan) has become an aging society, more specifically a super aging society in which the aging rate, which is a ratio of population of people aged 65 or older with respect to the total population, exceeds 21% due to the improvement of the standard of living, improvement of the hygienic environment, improvement of medical standards, and so forth accompanied by the high economic growth after the war. In addition, while the population of elderly people aged 65 or older was about 25.56 million in the total population of about 127.65 million in 2005, there is an estimate that the population of elderly people will be about 34.56 million in the total population of about 124.11 million. In such an aging society, increase in people requiring nursing and people requiring care (people requiring nursing and the like) who require nursing and care due to illness, injury, aging, or the like is expected to be greater than increase in people requiring nursing and the like in a normal society that is not an aging society. Further, our country is also a society with a declining birth rate in which, for example, the total fertility rate is 1.43 in 2013. Therefore, elderly-elderly nursing in which the care of an elderly person who requires nursing and the like is taken by an elderly family member (spouse, son/daughter, sibling) has also occurred.

People requiring nursing and the like enter facilities such as hospitals and welfare facilities for the elderly (according to Japanese law, short-term in-patient facilities for the elderly, nursing homes for the elderly, intensive care homes for the elderly, and the like), and receive nursing and care. In such facilities, some situations in which, for example, people requiring nursing and the like get injured by falling off beds or falling down while walking, or sneak out of beds and wander about may occur. It is necessary to solve such situations as quickly as possible. In addition, when such situations are left unsolved, such situations may develop into more serious situations. Therefore, in the facilities described above, nurses, care workers, and the like patrol regularly to confirm the safety and state of the people requiring nursing and the like.

However, increase in the number of nurses and the like does not keep up with increase in the number of people requiring nursing and the like, and thus the field of nursing and the field of care work are in chronic shortage of labor. Further, in periods of semi-night shift and night shift, the number of nurses, care workers, and the like is small compared with the period of day shift. Therefore, workload for each person is large and thus reduction of the workload is demanded. In addition, the facilities described above are no exception for the situation of elderly-elderly nursing described above, and elderly nurses and the like taking care of elderly people requiring nursing is often seen. Since physical strength generally declines when getting old, the load of nursing and the like becomes heavy for the elderly nurses and the like compared with for young nurses and the like, and the movement and decision making thereof also become slow even in the case where the elderly nurses are in good health.

In order to reduce the shortage of labor and the load on nurses and the like, technology that complements nursing work and care work is desired. Therefore, in recent years, monitored person monitoring technology of monitoring (monitoring) a monitored person such as a person requiring nursing or the like that is a monitored subject to be monitored has been studied and developed. In addition, such a device is useful for watching over a so-called single-living person who lives alone.

As an example of such a device, a falling-down detection system is disclosed in Patent Literature 1. The falling-down detection system disclosed in Patent Literature 1 includes a distance image sensor that detects a distance value of each pixel in a prescribed detection area, and a falling-down detection apparatus that detects falling-down of a person on the basis of the distance value of each pixel detected by the distance image sensor, and the falling-down detection apparatus sets a rectangular parallelepiped based on an outer shape of the person detected by the distance image sensor and detects falling-down of the person on the basis of an aspect ratio of the rectangular parallelepiped. Further, the distance image sensor obtains the distance value of each pixel by scanning laser light in a two-dimensional region and receiving the laser light reflected on an object by a two-dimensional scanner. In addition to this, sensors capable of obtaining three-dimensional information such as a stereo camera and a sensor including an LED and a CMOS are disclosed as examples of the distance image sensor.

By the way, in the falling-down detection system disclosed in Patent Literature 1 described above, the falling-down detection apparatus sets the rectangular parallelepiped based on the outer shape of the person detected by the distance image sensor and detects falling-down of the person on the basis of the aspect ratio of the rectangular parallelepiped. Therefore, in the case where, for example, a part of the body such as a foot is blocked from the distance image sensor by, for example, furniture such as a table or a chair, the setting of the rectangular parallelepiped becomes inaccurate, and the falling-down detection apparatus erroneously detects falling-down of the person. Therefore, in order to cancel the blockage, a method of detecting the distance value of each pixel in the detection area from plural angles by using plural distance image sensors can be considered. However, in this method, since plural detection sensors are used, the cost increases.

In addition, in the case where the person spreads arms, falling-down of the person cannot be detected on the basis of the aspect ratio of the rectangular parallelepiped because this case is not considered in the falling-down detection system disclosed in Patent Literature 1 described above.

### CITATION LIST

### Patent Literature

Patent Literature 1: JP 2014-16742 A

### SUMMARY OF INVENTION

The present invention has been made in consideration of the above circumstances, and an object thereof is to provide a posture detection device and a posture detection method that can determine a posture, for example, fall-down or fall-off, of a monitoring target more precisely with a simpler configuration.

In a posture detection device and a posture detection method according to the present invention, an image of a prescribed detection area is acquired by an image acquisition unit, a head portion is extracted from the acquired image of the detection area, a prescribed parameter for the extracted head portion is determined, and whether a prescribed posture is taken is determined on the basis of the determined parameter. Accordingly, in the posture detection device and the posture detection method according to the present invention, the posture of a monitored subject, can be determined more precisely with a simpler configuration by using a prescribed parameter for a head portion that is less likely to be blocked even in the case of a single image acquisition unit.

The object, feature, and merit described above and other objects, features, and merits of the present invention will be revealed in the detailed description below and attached drawings.

### BRIEF DESCRIPTION OF DRAWINGS

FIG. 1 is a block diagram illustrating a configuration of a posture detection device in an exemplary embodiment.

FIG. 2 is a diagram for description of an installation state of an image acquisition unit in the posture detection device.

FIG. 3 is a flowchart illustrating an operation of the posture detection device.

FIG. 4 is a diagram illustrating a fall-down/fall-off determination table in a third modification embodiment.

FIG. 5 is a diagram for description of a relationship between an image of a detection area and determination regions in the third modification embodiment.

FIG. 6 is a diagram for description of a relationship between an image of a detection area and determination regions of respective threshold values in a fourth modification embodiment.

FIG. 7 is a diagram for description of a relationship between an image of a detection area and determination regions for respective fall-down/fall-off determination in a fifth modification embodiment.

FIG. 8 is a diagram for description of a positional relationship between a head portion and a trunk in a sixth modification embodiment.

### DESCRIPTION OF EMBODIMENTS

An embodiment of the present invention will be described below in detail with reference to drawings. It is noted that elements assigned with the same reference sign in the drawings are the same elements, and the description thereof will be omitted as appropriate. In the present description, reference signs whose suffices have been omitted will be used for indicating elements collectively, and reference signs with suffices will be used for indicating individual elements.

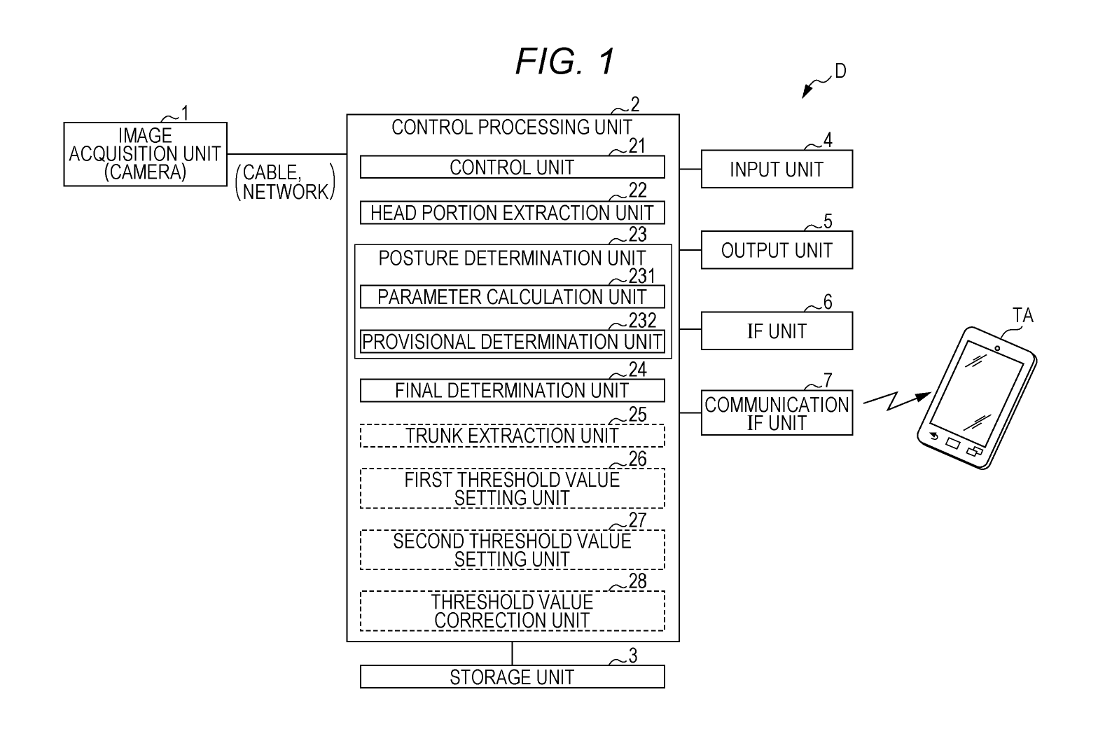

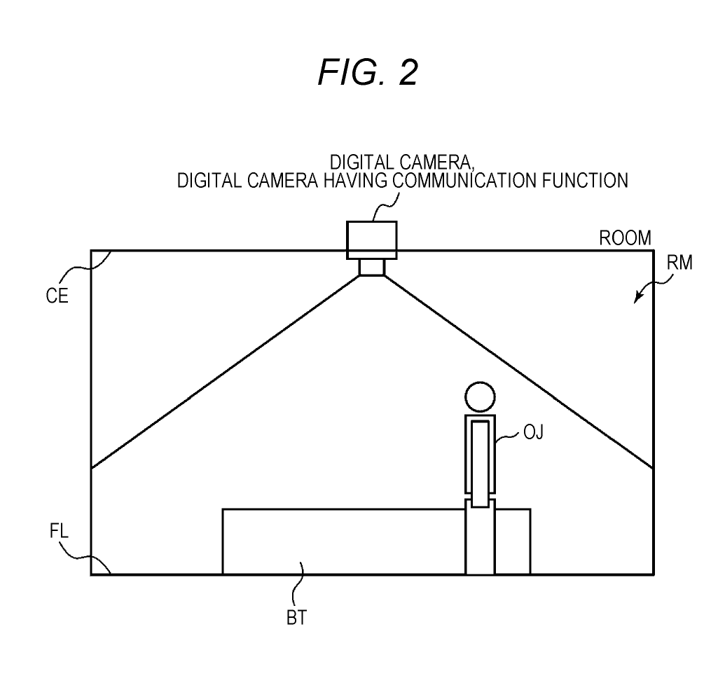

FIG. 1 is a block diagram illustrating a configuration of a posture detection device in an exemplary embodiment. FIG. 2 is a diagram for description of an installation state of an image acquisition unit in the posture detection device.

The posture detection device of the present exemplary embodiment acquires an image of a detection area, and determines, on the basis of the acquired image, whether a monitored subject (monitored person, watched person, or subject person) to be monitored, for example, a nursed person, a patient, or a single-living person, is in a prescribed posture that has been preset. Such a posture detection device D includes, for example, an image acquisition unit 1 and a control processing unit 2 including a head portion extraction unit 22 and a posture determination unit 23 as illustrated in FIGS. 1 and 2, and further includes a storage unit 3, an input unit 4, an output unit 5, an interface unit (IF unit) 6, and a communication interface unit (communication IF unit) 7 in an example illustrated in FIG. 1.

The image acquisition unit 1 is a device that is connected to the control processing unit 2 and acquires an image of a prescribed detection area under control of the control processing unit 2. The prescribed detection area is, for example, a space in which a monitored subject is normally present or is expected to be normally present. The image acquisition unit 1 is, for example, a communication interface such as a data communication card or a network card that receives, in the case of capturing an image of the detection area with a digital camera having a communication function such as a so-called web camera (web camera), a communication signal including the image of the detection area from the web camera via a network. In this case, the image acquisition unit 1 may be the communication IF unit 7, and also can be used as the communication IF unit 7. In addition, for example, the image acquisition unit 1 may be a digital camera that is connected to the control processing unit 2 via a cable. Such a digital camera includes, for example, an imaging optical system that forms an optical image of the detection area on a prescribed imaging plane, an image sensor that is disposed such that a light receiving surface coincides with the imaging plane and that converts the optical image of the detection area into an electrical signal, an image processing unit that generates an image (image data) of the detection area by subjecting an output from the image sensor to image processing, and so forth. It is noted that the digital camera having a communication function further includes a communication interface unit connected to the image processing unit for communicating a communication signal between the digital camera and the posture detection device D via a network. Such a digital camera (including the digital camera having a communication function) is disposed such that an imaging direction coincides with a direction appropriate for the detection area. For example, in the present exemplary embodiment, the digital camera is disposed at a center position, in a room (living room) RM in which the monitored subject is present, on a ceiling CE positioned sufficiently higher than the height of the monitored subject OJ such that the imaging direction thereof (optical axis direction of the imaging optical system) coincides with the vertical direction (normal direction of a horizontal ceiling surface of the ceiling) as illustrated in FIG. 2 such that the monitored subject is not blocked in the sight of the digital camera. In the example illustrated in FIG. 2, a state in which the monitored subject OJ is standing beside a bed BT disposed in a substantially center region of the room RM is illustrated. It is noted that although the digital camera may be a camera of visible light, the digital camera alternatively may be a camera of infrared light combined with an infrared light projector that projects infrared light such that an image can be also captured in the dark, for example, during night time.

The input unit 4 is a device that is connected to the control processing unit 2 and inputs various commands, for example, commands instructing monitoring, and various data required for monitoring, for example, the name of the monitored subject, into the posture detection device D, and examples thereof include a keyboard and a mouse. The output unit 5 is a device that is connected to the control processing unit 2 and outputs commands and data input from the input unit 4, determination results (for example, information indicating that the monitored subject is in a prescribed posture) of determination by the posture detection device D, and so forth under the control of the control processing unit 2, and examples thereof include display devices such as CRT displays, LCDs, and organic EL displays, and printing devices such as printers.

It is noted that a touch panel may be constituted by the input unit 4 and the output unit 5. In the case of constituting this touch panel, the input unit 4 is a position input device that employs, for example, a resistance film system or a capacitance system, and detects and inputs an operation position, and the output unit 5 is a display device. In this touch panel, the position input device is provided on a display screen of the display device, and one or plural candidates of input items that can be input are displayed on the display device. In the case where a user touches a display position at which an input item that is desired to be input, the position thereof is detected by the position input device, and the displayed item displayed at the detected position is input in the posture detection device D as an operation input item of the user. With such a touch panel, the user is likely to instinctively understand an input operation, and thus the posture detection device D that can be used easily by the user can be provided.

The IF unit 6 is a circuit that is connected to the control processing unit 2 and performs input and output of data between the IF unit 6 and an external device under the control of the control processing unit 2, and examples thereof include an interface circuit of RS-232C, which is a serial communication system, an interface circuit employing a Bluetooth (registered trademark) standard, an interface circuit that performs infrared light communication employing a standard such as infrared data association (IrDA), and an interface circuit employing a universal serial bus (USB) standard.

The communication IF unit 7 is a communication device connected to the control processing unit 2 for performing communication with a communication terminal apparatus TA via a net (network) such as a LAN, a telephone network, and a data communication network through wired connection or wireless connection under the control of the control processing unit 2. The communication IF unit 7 generates, in accordance with a communication protocol used in the network, a communication signal including data to be transmitted that has been input from the control processing unit 2, and transmits the generated communication signal to the communication terminal apparatus TA via the network. The communication IF unit 7 receives a communication signal from another device such as the communication terminal apparatus TA via the network, takes out data from the received communication signal, converts the taken-out data into data of a format that can be processed by the control processing unit 2, and outputs the data to the control processing unit 2.

The storage unit 3 is a circuit that is connected to the control processing unit 2 and stores various prescribed programs and various prescribed data under the control of the control processing unit 2. The various prescribed programs include, for example, a control processing program such as a posture detection program for detecting a prescribed posture of a monitored subject in an image of the detection area. The various prescribed data include a threshold value th for determining whether the prescribed posture is taken and so forth. The storage unit 3 includes, for example, a read only memory (ROM) that is a nonvolatile storage device, an electrically erasable programmable read only memory (EEPROM) that is a rewritable nonvolatile storage device, and so forth. Further, the storage unit 3 also includes a random access memory (RAM) that stores data and the like generated during execution of the prescribed program and serves as a working memory of a central processing unit (CPU), and so forth. It is noted that the storage unit 3 may include a hard disk having a relatively large capacity.

The control processing unit 2 is a circuit for controlling each component of the posture detection device D in accordance with the function of each component, and detecting the prescribed posture of the monitored subject. The control processing unit 2 includes, for example, a central processing unit (CPU) and peripheral circuits of the CPU. As a result of the control processing program being executed, a control unit 21, the head portion extraction unit 22, the posture determination unit 23, and a final determination unit 24 are functionally configured in the control processing unit 2, and a parameter calculation unit 231 and a provisional determination unit 232 are functionally configured in the posture determination unit 23.

The control unit 21 is a unit for controlling each component of the posture detection device D in accordance with the function of each component.

The head portion extraction unit 22 extracts a head portion (an image region in an image in which a head portion is captured, or an image of a head portion) from an image of the detection area acquired by the image acquisition unit 1. A known image processing technique is used for extraction of the head portion. For example, the shape of a head portion is assumed to be an elliptical shape, the image of the detection area is transformed via so-called generalized Hough transform, and an elliptical shape, that is, a head portion, in the image of the detection area is thereby extracted. Such an image processing technique is disclosed in, for example, a document “Murakami, Makoto ‘The Effective Representation and Extraction Methods for Human Face Recognition’ (March 2003), Waseda University”. Alternatively, a head portion may be extracted from the image of the detection area via, for example, template matching using a head portion shape such as an outline shape of the head portion or an elliptical or circular shape that is a schematic shape thereof as a template that has been prepared in advance, or via a method of fitting a closed curve such as Snake. In addition, from the viewpoint of improving the precision of extraction, color information such as a skin color and a black color, movement information for determination of being a person or not from presence of movement, and so forth may be used in combination with these methods. Alternatively, from the viewpoint of shortening image processing time, the color information, movement information and so forth may be used to restrict a region on which image processing is to be performed to a region with a high possibility of a person being present therein in the image of the detection area. The head portion extraction unit 22 notifies the extracted head portion (image region of head portion) to the posture determination unit 23.

The posture determination unit 23 determines a prescribed parameter for the head portion extracted by the head portion extraction unit 22, and determines whether a prescribed posture that has been defined in advance is taken on the basis of the determined parameter. More specifically, the posture determination unit 23 determines whether the prescribed posture is taken on the basis of whether or not the prescribed parameter for the head portion extracted by the head portion extraction unit 22 is equal to or larger than a prescribed threshold value th. In the present exemplary embodiment, the posture determination unit 23 functionally includes the parameter calculation unit 231 and the provisional determination unit 232.

The parameter calculation unit 231 determines the prescribed parameter for the head portion extracted by the head portion extraction unit 22. An appropriate parameter with which the posture of the monitored subject can be determined may be used as the prescribed parameter. For example, in the case of determining whether fall-down/fall-off is taken, the height of the head portion can be used as the parameter because the height of the head portion differs between the posture of fall-down/fall-off and other postures such as a standing position and a sitting position. In addition, for example, in the case of determining whether the monitored subject is in a standing position, whether the monitored subject is in a sitting position, or whether the monitored subject is in fall-down/fall-off, the height of the head portion can be also used because the height of the head portion differs between each posture of the standing position, sitting position, and fall-down/fall-off also in this case. In the case where an image of the detection area is captured by viewing the detection area from above in a height direction of the monitored subject, the size of the head portion in the image (the length of a shorter side of an image region in which the head portion is captured) is a size corresponding to the height of the head portion. That is, at the same position on a flat surface, the size of the head portion appears bigger in the image when the height of the head portion is higher. Therefore, in each of the cases described above, the size of the head portion can be used as the parameter. That is, the height of the head portion can be estimated by using the size of the head portion as the parameter, and the posture of the monitored subject such as the standing position, sitting position, and fall-down/fall-off can be determined on the basis of the estimated height of the head portion.

The provisional determination unit 232 determines whether the prescribed posture is taken on the basis of whether or not the prescribed parameter for the head portion determined by the parameter calculation unit 231 is equal to or larger than the prescribed threshold value th. According to this, whether the prescribed posture is taken can be determined easily by just determining whether or not the parameter is equal to or larger than the threshold value th. More specifically, for example, in the case of determining whether fall-down/fall-off is taken by using the height of the head portion as the parameter, a height of the head portion with which the posture of fall-down/fall-off can be distinguished from other postures such as the standing position and the sitting position is preset as the prescribed threshold value (first threshold value, or fall-down/fall-off determination head portion height threshold value) th1. Alternatively, in the case where it is desired that just a posture of having completely fall-down is detected, the height of the bed BT may be set as the threshold value th1. In addition, for example, in the case of determining whether the monitored subject is in the standing position, whether the monitored subject is in the sitting position, or whether the monitored subject is in fall-down/fall-off by using the height of the head portion as the parameter, a height of the head portion with which the posture of the standing position can be distinguished from the posture of the sitting position is preset as the prescribed threshold value (second-first threshold value, or standing position/sitting position determination head portion height threshold value) th21, and a height of the head portion with which the posture of the sitting position can be distinguished from the posture of fall-down/fall-off is preset as the prescribed threshold value (second-second threshold value, or sitting position/fall-down/fall-off head portion height threshold value) th22. In the case where the size of the head portion is used as the parameter, the respective threshold values th1, t21, and th22 are similarly preset by replacing the height of the head portion by the size of the head portion. The respective threshold values th1, th21, and th22 may be appropriately set by preparing plural samples in advance and performing statistical processing.

Here, in the case of setting the threshold values th1 and th22 for determining fall-down/fall-off, the height of the sitting position varies depending on the height of the standing position, that is, the stature. Therefore, it is preferable that the threshold values th1 and th22 are set on the basis of the height of the standing position. By setting the threshold values th1 and th22 so as to be lower than the height of the sitting position on the basis of the height of the standing position (stature), such a posture detection device D becomes capable of determining whether the posture of the monitored subject is fallen down/fallen off. In addition, it is preferable that the threshold values th1 and th22 are set on the basis of the height of the sitting position. By setting the threshold values th1 and th22 so as to be lower than the height of the sitting position on the basis of the height of the sitting position, such a posture detection device D becomes capable of determining whether the posture of the monitored subject is fallen down/fallen off.

Further, the provisional determination unit 232 notifies the determination result to the final determination unit 24 as the result of determination of the posture determination unit 23.

Here, in the present exemplary embodiment, the image acquisition unit 1 acquires plural images of the detection area at mutually different times, the head portion extraction unit 22 extracts a head portion from each of the plural images of the detection area acquired by the image acquisition unit 1, and the posture determination unit 23 determines, for each of the plural images of the detection area acquired by the image acquisition unit 1, whether a prescribed posture is taken on the basis of a prescribed parameter for the head portion extracted by the head portion extraction unit 22.

Further, the final determination unit 24 makes final determination of whether the prescribed posture is taken on the basis of plural determination results of determination by the posture determination unit 23. For example, the final determination unit 24 makes final determination that the prescribed posture is taken in the case where it is determined that the prescribed posture is taken a prescribed number of consecutive times (that is, the whole time during a prescribed period of time) in the plural determination results of determination by the posture determination unit 23. In the case where the final determination that the prescribed posture is taken is made, the final determination unit 24 notifies this fact to the control unit 21. The control unit 21 outputs information indicating that the posture of the monitored subject is finally the prescribed posture when receiving the notification indicating that the posture of the monitored subject is finally the prescribed posture from the final determination unit 24.

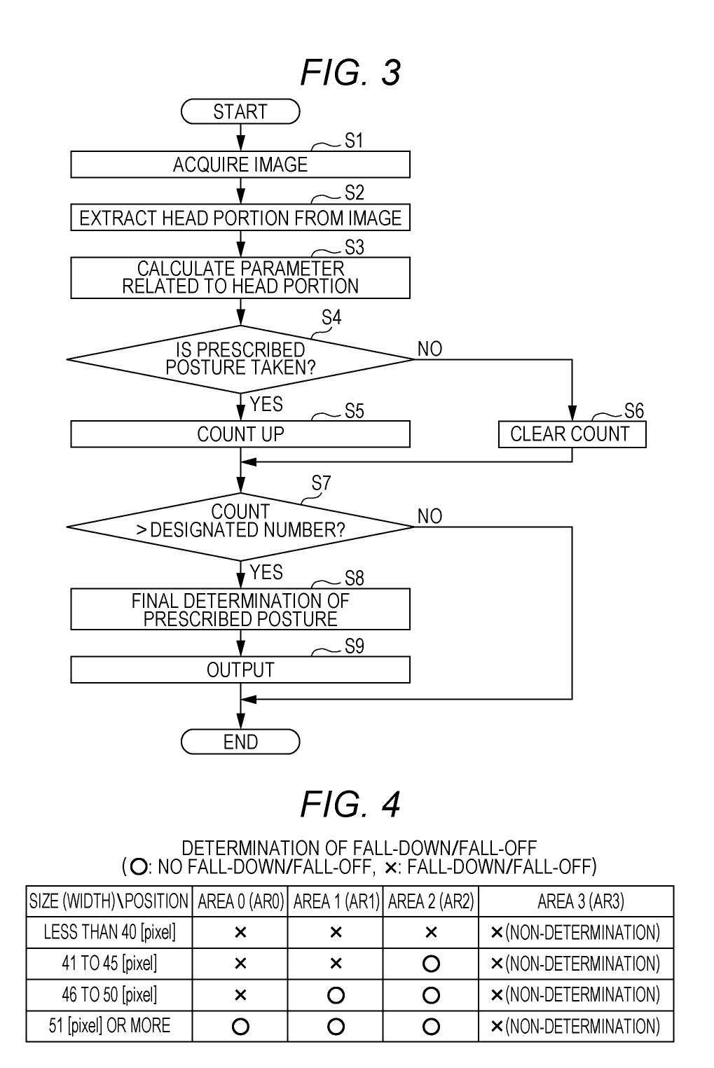

Next, the operation of the posture detection device D will be described. FIG. 3 is a flowchart illustrating an operation of the posture detection device in the exemplary embodiment. In such a posture detection device D, when a user (operator) turns on a power switch whose illustration is omitted, the control processing unit 2 performs initialization of each component as necessary, and, as a result of execution of the control processing program, the control unit 21, the head portion extraction unit 22, the posture determination unit 23, and the final determination unit 24 are functionally configured in the control processing unit 2, and the parameter calculation unit 231 and the provisional determination unit 232 are configured in the posture determination unit 23.

In the determination of the prescribed posture that has been defined in advance, first, an image of the detection area is acquired by the image acquisition unit 1, and the acquired image of the detection area is output from the image acquisition unit 1 to the control processing unit 2 (S1) in FIG. 3.

Next, a head portion (image region in which a head portion is captured) is extracted by the head portion extraction unit 22 of the control processing unit 2 from the image of the detection area acquired by the image acquisition unit 1, and the extracted head portion is notified to the posture determination unit 23 of the control processing unit 2 (S2).

Next, a prescribed parameter for the head portion extracted by the head portion extraction unit 22, for example, the size of the head portion, is determined by the parameter calculation unit 231 of the posture determination unit 23, and the determined parameter (size of the head portion in this example) is notified from the parameter calculation unit 231 to the provisional determination unit 232 of the posture determination unit 23 (S3).

Next, whether a prescribed posture defined in advance is taken is determined by the provisional determination unit 232 on the basis of the parameter (size of the head portion in this example) determined by the parameter calculation unit 231 (S4). More specifically, for example, the provisional determination unit 232 determines whether or not the size of the head portion determined by the parameter calculation unit 231 is equal to or larger than the threshold value th1 for determination of fall-down/fall-off, and thereby determines whether fall-down/fall-off is taken. In the case where the size of the head portion is equal to or larger than the threshold value th1 as a result of this determination, the provisional determination unit 232 determines that fall-down/fall-off is not taken, that is, the prescribed posture is not taken (No), and notifies the determination result indicating that the prescribed posture is not taken to the final determination unit 24, and a process S6 is performed. In contrast, in the case where the size of the head portion is not equal to or larger than the threshold value th1 as a result of the determination, the provisional determination unit 232 determines that fall-down/fall-off is taken, that is, the prescribed posture is taken (Yes), and notifies the determination result indicating that the prescribed posture is taken to the final determination unit 24, and a process S5 is performed.

In the process S5, when the determination result indicating that the prescribed posture is taken is received, the final determination unit 24 causes a counter CT that counts the number of determination results indicating that the prescribed posture is taken to count up (CT←CT+1), and a process S7 is performed.

In contrast, in the process S6, when the determination result indicating that the prescribed posture is not taken is received, the final determination unit 24 causes the counter CT to clear the count (CT←0), and the process S7 is performed. It is noted that in the case where the provisional determination unit 232 makes erroneous determination, since the counter CT is cleared after one erroneous determination in the process S6, the final determination unit 24 may cause the counter CT to count down (CT←CT−1) instead of clearing the counter CT in the process S6.

In the process S7, the final determination unit 24 determines whether the counter CT exceeds a preset designated number. The designated number is the number of determination results indicating that the prescribed posture is taken from the provisional determination unit 232 necessary for making final determination that the prescribed posture is taken, and is set to, for example, an appropriate number such as 5 or 10 in consideration of a time interval of output of one determination result from the provisional determination unit 232.

In the case where the counter CT does not exceed the designated number as a result of this determination (No), the determination processing of this time is finished and next determination processing is performed. That is, the processes described above are performed starting from the process S1.

In contrast, in the case where the counter CT exceeds the designated number (Yes) as a result of the determination, the final determination unit 24 makes final determination that the posture of the monitored subject is the prescribed posture, and the final determination unit 24 notifies the fact that it has been finally determined that the posture of the monitored subject is the prescribed posture to the control unit 21 (S8). Then, after receiving the notification, the control unit 21 outputs information indicating that the posture of the monitored subject is finally the prescribed posture when receiving the notification indicating that the posture of the monitored subject is finally the prescribed posture from the final determination unit 24 (S9). For example, the control unit 21 outputs information indicating that the posture of the monitored subject is finally the prescribed posture to the output unit 5. In addition, for example, the control unit 21 transmits a communication signal (posture notification signal) including the information indicating that the posture of the monitored subject is finally the prescribed posture to the communication terminal apparatus TA via the communication IF unit 7. When receiving the posture notification signal, the communication terminal apparatus TA displays the information indicating that the posture of the monitored subject is finally the prescribed posture on a display device thereof (liquid crystal display, organic EL display, or the like). Then, the determination processing of this time is finished, and next determination processing is performed. That is, the processes described above are performed starting from the process S1.

As described above, in the posture detection device D and the posture detection method employed for this in the present exemplary embodiment, the image acquisition unit 1 acquires an image of a detection area, the head portion extraction unit 22 extracts a head portion (image region in which a head portion is captured in the image, or an image of a head portion) from the image of the detection area, and the posture determination unit 23 determines a prescribed posture of a monitored subject (monitored person, watched person, or subject person) related to the head portion on the basis of a prescribed parameter for the head portion. Accordingly, in the posture detection device D and the posture detection method employed for this in the present exemplary embodiment, the posture of the monitored subject, for example, falling down or falling off, can be determined more precisely with a simpler configuration of using the single image acquisition unit 1 and by using a prescribed parameter for a head portion that is less likely to be blocked. Since the posture of spreading arms or the like does not affect the parameter for the head portion, the posture of the monitored subject can be determined more precisely. Since the posture of the monitored subject can be determined from just one image of the detection area, the posture detection device D and the posture detection method employed for this in the present exemplary embodiment can be realized by hardware with a relatively low information processing performance.

In the posture detection device D and the posture detection method employed for this in the present exemplary embodiment, the final determination unit 24 makes final determination whether the prescribed posture is taken on the basis of plural determination results determined by the posture determination unit 23, and thus the posture of the monitored subject can be determined more precisely.

In the posture detection device D and the posture detection method employed for this in the present exemplary embodiment, in the case where the image acquisition unit 1 is a camera disposed on the ceiling CE, the monitored subject OJ captured in the image of the detection area is less likely to be blocked by furniture or the like disposed in the room RM, and the posture of the monitored subject OJ can be determined more precisely.

It is noted that although the threshold values th1, th21, and th22 are set by performing statistical processing by using plural samples and the posture detection device D is configured as a general-purpose machine in the exemplary embodiment described above, the posture detection device D may further functionally include a first threshold value setting unit 26 that sets the threshold values th1, th21, and th22 for each subject person in the control processing unit 2 as indicated by broken lines in FIG. 1 (first modification embodiment). In this case, the user (operator) inputs the threshold values th1, th21, and th22 corresponding to the monitored subject through the input unit 4, and, when receiving the threshold values th1, th21, and th22 corresponding to the monitored subject from the input unit 4, the first threshold value setting unit 26 stores the threshold values th1, th21, and th22 in the storage unit 3 and sets the threshold values th1, th21, and th22. The provisional determination unit 232 of the posture determination unit 23 determines whether the prescribed posture is taken by using the threshold values th1, th21, and th22 corresponding to the monitored subject stored in the storage unit 3. In addition, in this case, although the threshold values th1, th21, and th22 themselves (the very values) corresponding to the monitored subject may be input through the input unit 4, the height of the standing position (stature) (or the height of the sitting position) of the monitored subject may be input through the input unit 4, and the first threshold value setting unit 26 may determine the threshold values th1, th21, and th22 from the height of the standing position (or the height of the sitting position) of the monitored subject received by the input unit 4 (convert the height of the standing position or the sitting position into the threshold values th1, th21, and th22), store the threshold values th1, th21, and th22 in the storage unit 3, and set the threshold values th1, th21, and th22. Since such a posture detection device D further includes the first threshold value setting unit 26 and can set the threshold values th1, th21, and th22 in correspondence with the monitored subject, customization can be performed in accordance with the monitored subject (for each monitored person), and thus the posture of the monitored subject can be determined even more precisely.

In addition, in the exemplary embodiment described above, the image acquisition unit 1 may acquire plural images of the detection area at mutually different times, and the posture detection device D may further functionally include a second threshold value setting unit 27 that sets the threshold values th1, th21, and th22 on the basis of the plural images acquired by the image acquisition unit 1 in the control processing unit 2 as indicated by broken lines in FIG. 1 (second modification embodiment). In this case, as preliminary processing for respective processes S1 to S9 for determining the posture, the actual behavior of the monitored subject in the detection area may be observed by acquiring plural images of the detection area at mutually different times by the image acquisition unit 1, each prescribed parameter related to the head portion may be determined from the each of the plural images by the second threshold value setting unit 27, the average value or the minimum value of each parameter may be determined after removing outliers (noise), the threshold values th1, th21, and th22 may be determined from the determined value and stored in the storage unit 3 (the determined value may be converted into the threshold values th1, th21, and th22), and the threshold values th1, th21, and th22 may be thereby set. Since such a posture detection device D sets the threshold values th1, th21, and th22 by the second threshold value setting unit 27 on the basis of plural images of the detection area at mutually different times, the threshold values th1, th21, and th22 can be set automatically for each subject person. In particular, even in the case where the posture of the standing position or walking is different from a healthy person as a result of, for example, a bent back, the threshold values th1, th21, and th22 can be set automatically in consideration of such a personal circumstance.

In addition, in these embodiments (including the first and second modification embodiments) described above, the posture detection device D may further functionally include a threshold value correction unit 28 that corrects the threshold values th1, th21, and th22 that are preset or set by the first threshold value setting unit 26 or the second threshold value setting unit 27 in the control processing unit 2 as indicated by broken lines in FIG. 1 (third modification embodiment and fourth modification embodiment).

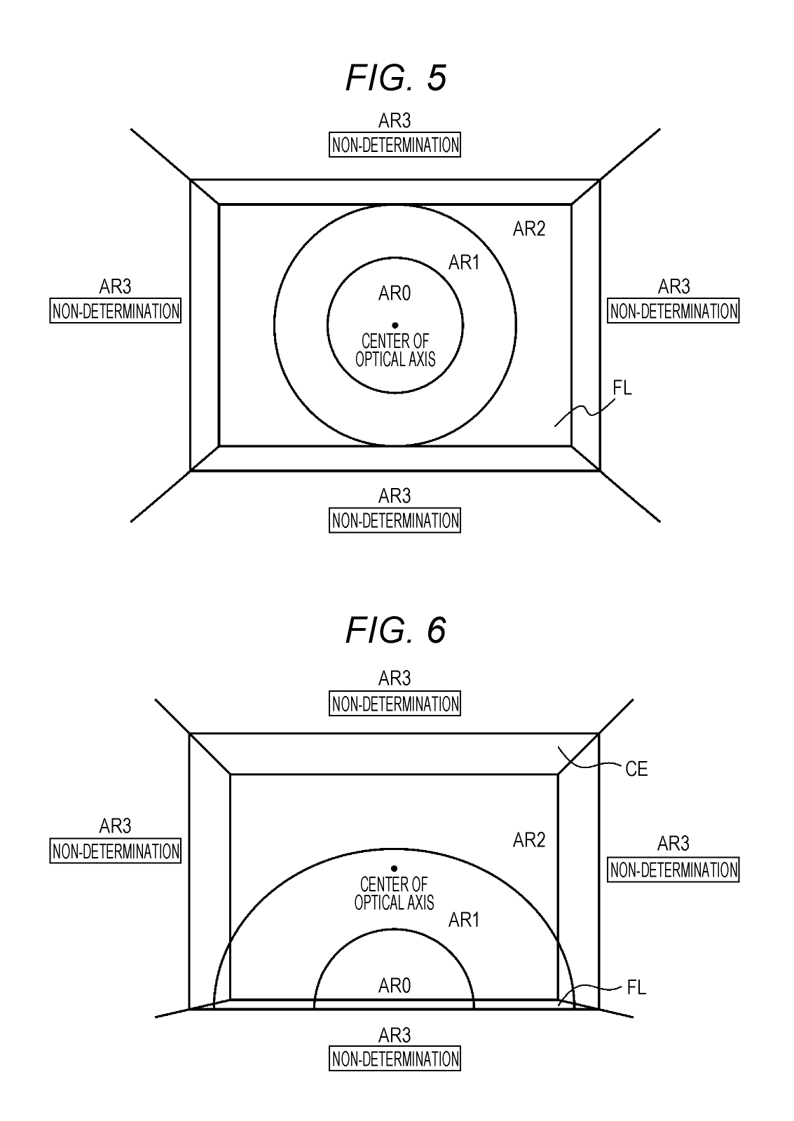

FIG. 4 is a diagram illustrating a fall-down/fall-off determination table in the third modification embodiment. FIG. 5 is a diagram for description of a relationship between an image of a detection area and determination regions in the third modification embodiment. FIG. 6 is a diagram for description of a relationship between an image of a detection area and determination regions of respective threshold values in the second modification embodiment.

As illustrated in FIG. 2, in the case where the digital camera is disposed at a center position on the ceiling CE, the size of the head portion is substantially proportional to the height of the head portion in a region around an optical axis in the image or in the case where the angle of view of the digital camera is relatively small, and thus the prescribed posture of the monitored subject can be determined from the size of the head portion. That is, in the case where the digital camera is not tilted with respect to the floor FL, the lens is not distorted, the height of the head portion is represented by C (m), the height of the ceiling CE is represented by H (m), the size of the head portion on the surface of the floor FL is represented by Sh (pixel), and the size (width) of the head portion determined by the parameter calculation unit 231 is represented by Si (pixel), C=H×(1−(Sh/Si) holds. It is noted that Sh may be calculated from the specification of the digital camera and the position at which the digital camera is installed, or may be measured.

However, at a peripheral region in the image or in the case where the angle of view of the digital camera is relatively large, the size of the head portion and the height of the head portion are not necessarily in a proportional relationship. Thus, the threshold value correction unit 28 corrects the threshold values th1, th21, and th22 to be used by the provisional determination unit 232 in accordance with the position of the head portion in the image (position in the image in which the head portion is captured) such that the deviation from the proportional relationship between the size of the head portion and the height of the head portion is canceled. It is noted that the aberration of the imaging optical system may be taken into account for this correction.

For this correction, although a function formula representing a relationship between the position of the head portion in the image and a correction value may be stored in the storage unit 3 and the function formula may be used by the provisional determination unit 232, a table illustrated in FIG. 4 may be stored in the storage unit 3 and the table may be used by the provisional determination unit 232. In the table illustrated in FIG. 4, positions of the head portion in the image are divided into four areas of first to fourth determination areas AR0 to AR3 as illustrated in FIG. 5, and a different threshold value this set for each of the first to fourth determination areas AR0 to AR3. That is, the threshold value th1 for fall-down/fall-off in the first determination area AR0 which is a region in a circle having a prescribed first radius and the optical axis as the center and in which the size of the head portion and the height of the head portion are substantially in a proportional relationship is, for example, 51 [pixel], and, in the case where the position of the head portion extracted by the head portion extraction unit 22 is in the first determination area AR0, the posture of the monitored subject is determined as no fall-down/fall-off (◯) (not fall-down/fall-off) when the size of the head portion (length of a shorter side of the image region in which the head portion is captured) calculated by the parameter calculation unit 231 is equal to or larger than 51 [pixel], and the posture of the monitored subject is determined as fall-down/fall-off (x) when the size of the head portion calculated by the parameter calculation unit 231 is smaller than 51 [pixel]. The threshold value th1 for fall-down/fall-off in the second determination area AR1 which is concentric with the first determination area AR0, exceeds the first determination area AR0, and is a region in a circle having a prescribed second radius (>first radius) and the optical axis as the center is, for example, 46 [pixel], and, in the case where the position of the head portion extracted by the head portion extraction unit 22 is in the second determination area AR1, the posture of the monitored subject is determined as no fall-down/fall-off (◯) (not fall-down/fall-off) when the size of the head portion calculated by the parameter calculation unit 231 is equal to or larger than 46 [pixel], and the posture of the monitored subject is determined as fall-down/fall-off (x) when the size of the head portion calculated by the parameter calculation unit 231 is smaller than 46 [pixel]. The threshold value th1 for fall-down/fall-off in the third determination area AR2 which is a region that exceeds the second determination area AR1 and includes the floor FL and wall surfaces to a prescribed height is, for example, 41 [pixel], and, in the case where the position of the head portion extracted by the head portion extraction unit 22 is in the third determination area AR2, the posture of the monitored subject is determined as no fall-down/fall-off (o) (not fall-down/fall-off) when the size of the head portion calculated by the parameter calculation unit 231 is equal to or larger than 41 [pixel], and the posture of the monitored subject is determined as fall-down/fall-off (x) when the size of the head portion calculated by the parameter calculation unit 231 is smaller than 41 [pixel]. The second and third determination areas AR1 and AR2 are areas in which the size of the head portion and the height of the head portion are not in a proportional relationship, and, in this example, the areas are divided into two regions in accordance with the degree of deviation from the proportional relationship between the size of the head portion and the height of the head portion so as to perform correction more precisely. The fourth determination area AR3 that is a region exceeding the third determination area AR2 in the image is set as a non-determination area (area in which determination cannot be performed), and the threshold value th1 for the fourth determination area AR3 is not set. Since a different value of the threshold value this set for each determination area AR in this way, it becomes possible to perform determination in consideration of the change in the relationship between the size and height of the head portion depending on the position in the image. In addition, according to this, it becomes possible to perform determination in consideration of a specific area in which a bed or the like is present.

In addition, although the digital camera is disposed at the center position on the ceiling CE such that the imaging direction coincides with the vertical direction in the description above, in some case the digital camera may capture the image of the detection area via swing and tilt photographing method as illustrated in FIG. 6 depending on the disposed position of the digital camera or the set direction of the imaging direction. In such a case, as illustrated in FIG. 6, the shape of the determination areas may be changed appropriately in accordance with an imaging condition (camera characteristics), the threshold value for each determination area may be appropriately set, and the table may be thereby generated. In an example illustrated in FIG. 6, the digital camera is disposed at one corner on the upper side of the room RM with the imaging direction directed obliquely downward, the first determination area AR0 is set as a region in a semicircle having a prescribed third radius and a point on the floor FL right below the center of the optical axis as the center, the first determination area AR1 is set as a region that is concentric with the first determination area AR0, exceeds the first determination area AR0, and is in a semicircle having a prescribed fourth radius (>third radius) and the point on the floor FL right below the center of the optical axis as the center, the third determination area AR2 is set as a region that exceeds the second determination area AR1 and includes respective positions of a rear wall surface and a ceiling surface CE, a right wall surface, and a left wall surface continuous with the rear wall surface, and the fourth determination area AR3 is set as a region in the image that exceeds the third determination area AR2. The threshold value th1 is appropriately set for each of these first to third determination areas AR0 to AR2 in consideration of swing and tilt photographing as an imaging condition, the fourth determination area AR4 is set as a non-determination area (area in which determination cannot be performed), and the threshold value th1 for fall-down/fall-off is not set for the fourth determination area AR3.

Here, respective threshold values th1 for these first to third determination areas AR0 to AR2 are, for example, set as follows. First, a model of a head portion (head portion model) having a statistically standard size is prepared in advance, images of the head portion model whose size is known are captured by the digital camera for the respective determination areas AR0 to AR2 at heights with which whether fall-down/fall-off is taken is determined, the sizes (numbers of pixels) of the head portion model in the images are determined, and the determined sizes (numbers of pixels) of the head portion model in the images are set as the threshold values th1.

It is noted that although an example concerning the size of the head portion has been described above, the same applies to the height of the head portion. In addition, although the breaking of the proportional relationship between the size of the head portion and the height of the head portion is canceled by correcting the threshold values th1, th21, and th22 by the threshold value correction unit 28 in the above description, the image of the detection area acquired by the image acquisition unit 1, the head portion (image of the head portion) extracted by the head portion extraction unit 22, or the parameter for the head portion calculated by the parameter calculation unit 231 may be corrected so as to cancel the breaking of the proportional relationship between the size of the head portion and the height of the head portion.

In addition, the parameter in the embodiments (including the first to fourth modification embodiments) described above may further include the position of the head portion (fifth modification embodiment). That is, for example, the posture determination unit 23 determines the size and position of the head portion extracted by the head portion extraction unit 22 and determines whether the prescribed posture is taken on the basis of the determined size and position of the head portion. In addition, in another example, the posture determination unit 23 determines the height and position of the head portion extracted by the head portion extraction unit 22 and determines whether the prescribed posture is taken on the basis of the determined height and position of the head portion.

In the case where whether the prescribed posture that has been defined in advance is taken is determined by the posture determination unit 23, there may be a case where the prescribed posture does not occur depending on the position of the monitored subject. Conversely, there may be a case where there is a high possibility that the prescribed posture has occurred depending on the position of the monitored subject. For example, in the case where whether fall-down/fall-off is taken is determined by the posture determination unit 23 and the monitored subject is present on a bed, it is highly possible that the monitored subject is just lying on the bed and fall-down/fall-off is not taken even in the case where it is determined that fall-down/fall-off is taken in determination using the threshold value th1. Conversely, in the case where the position of the monitored subject is on the floor, it is highly possible that the monitored subject has fallen down/fallen off. Therefore, the position of the monitored subject is estimated from the position of the head portion, and the posture determination unit 23 can determine the posture of the monitored subject even more precisely by determining whether the prescribed posture is taken in consideration of the position of the head portion, that is, the position of the monitored subject, in addition to the size of the head portion or the height of the head portion as described above.

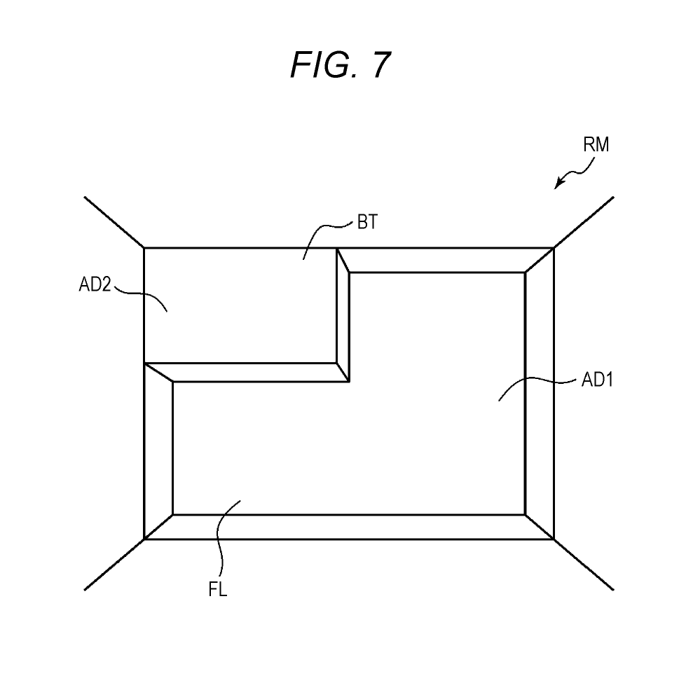

FIG. 7 is a diagram for description of a relationship between an image of a detection area and determination regions for respective fall-down/fall-off determination in the fifth modification embodiment. More specifically, in the case where the bed BT is placed in the room RM of the detection area as illustrated in FIG. 7, a region AD2 in the image corresponding to the bed BT is set as a determination area of non-determination, and, conversely, a region AD1 in the image corresponding to the floor FL is set as a determination area for determination and is stored in the storage unit 3. The posture determination unit 23 refers to the storage unit 3 before determining (or after determining) whether the prescribed posture is taken by using the size of the head portion or the height of the head portion, and determines whether the position of the head portion is in the determination area of non-determination. Alternatively, the region AD2 in the image corresponding to the bed BT may be included in the third determination area AR2 in the table illustrated in FIG. 4.

From this viewpoint, in the posture detection device D described above, the posture determination unit 23 preferably determines whether fall-down/fall-off serving as the prescribed posture is taken on the basis of whether the position of the head portion extracted by the head portion extraction unit 22 is on the floor. In the case where the position of the head portion is on the floor, it is highly possible that the posture of the monitored subject is fallen down/fallen off. Therefore, such a posture detection device D determines, by the posture determination unit 23, whether fall-down/fall-off serving as the prescribed posture is taken on the basis of whether the position of the head portion is on the floor, and thus can make determination of fall-down/fall-off more precisely.

In addition, from this viewpoint, in the posture detection device D described above, the posture determination unit 23 preferably determines whether fall-down/fall-off serving as the prescribed posture is taken on the basis of whether the position of the head portion extracted by the head portion extraction unit 22 is on the bed. In the case where the position of the head portion is on the bed, it is highly possible that the posture of the monitored subject is not fallen down/fallen off but is lying on the bed. Therefore, such a posture detection device D determines, by the posture determination unit 23, whether fall-down/fall-off serving as the prescribed posture is taken on the basis of whether the position of the head portion is on the bed, and thus can make determination of fall-down/fall-off more precisely. In other words, lying on the bed can be determined.

In addition, the parameter in the embodiments (including the first to fifth modification embodiments) described above may further include the orientation of the head portion (sixth modification embodiment). That is, for example, the posture determination unit 23 determines the size and orientation of the head portion extracted by the head portion extraction unit 22 and determines whether the prescribed posture is taken on the basis of the determined size and orientation of the head portion. In addition, in another example, the posture determination unit 23 determines the height and orientation of the head portion extracted by the head portion extraction unit 22 and determines whether the prescribed posture is taken on the basis of the determined height and orientation of the head portion. In addition, for example, the posture determination unit 23 determines the size, position, and orientation of the head portion extracted by the head portion extraction unit 22 and determines whether the prescribed posture is taken on the basis of the determined size, position, and orientation of the head portion. In addition, in another example, the posture determination unit 23 determines the height, position, and orientation of the head portion extracted by the head portion extraction unit 22 and determines whether the prescribed posture is taken on the basis of the determined height, position, and orientation of the head portion. Here, in the case where the angle that a median line connecting the center position of both eyes and the chin forms with the vertical direction is 0 degree, the face directs in the horizontal direction. The head portion directing sideways indicates a state in which the median line of the head portion forms an angle close to 90 degree with the vertical direction and the face directs in the horizontal direction. Accordingly, the parameter of orientation corresponds to the angle each of the face direction and the median line of the head portion forms with the vertical direction.

In the case where whether the prescribed posture that has been set in advance is taken is determined by the posture determination unit 23, there may be a case where the prescribed posture does not occur depending on the orientation of the head portion of the monitored subject. Conversely, there may be a case where there is a high possibility that the prescribed posture has occurred depending on the orientation of the head portion of the monitored subject. For example, in the case where whether fall-down/fall-off is taken is determined by the posture determination unit 23, it is highly possible that the monitored subject has not fallen down/fallen off but is squatting down when the orientation of the head portion, that is, the orientation of the face that can be determined from the orientation of the head portion directs straight to the front (in the horizontal direction), and, conversely, it is highly possible that the monitored subject has fallen down/fallen off when the orientation of the head portion, that is, the orientation of the face that can be determined from the orientation of the head portion directs sideways or upward. In addition, for example, in the case where the head portion is right under the digital camera and the orientation of the head portion is upward (in the case where the head portion has been extracted as not an elliptical shape but a substantially circular shape), it is not determined that the monitored subject has fallen down. Therefore, as described above, the posture determination unit 23 can determine the posture of the monitored subject even more precisely by determining whether the prescribed posture is taken in consideration also of the orientation of the head portion (that is, the orientation of the face).

In this case, a known image processing technique is used for extraction of the orientation of the head portion. The orientation of the face is extracted by the parameter calculation unit 231 via, for example, template matching using an outline shape of the head portion as a template that has been prepared in advance, template matching using a face shape constituted by, for example, feature points of the face such as eyes and a mouth, or Haal-like focusing on the feature points of the face, and thus the orientation of the head portion is determined. It is noted that the orientation of the head portion may be determined by the head portion extraction unit 22 instead of the parameter calculation unit 231. Further, the posture determination unit 23 determines whether the prescribed posture is taken by using the parameter including the orientation of the head portion. For example, in the case where the size of the head portion determined by the parameter calculation unit 231 is not equal to or larger than the threshold value th1 for determining whether fall-down/fall-off is taken, the posture determination unit 23 determines that fall-down/fall-off is not taken when the orientation of the head portion directs straight to the front (in the horizontal direction), and, in contrast, determines that fall-down/fall-off is taken when the orientation of the head portion is sideways or upward.

Here, with only the head portion extracted by the head portion extraction unit 22, a case in which it is difficult to determine the orientation of the head portion by the parameter calculation unit 231 may occur. Therefore, the posture detection device D may further include a trunk extraction unit 25 that extracts, from the image of the detection area acquired by the image acquisition unit 1, a trunk corresponding to the head portion extracted by the head portion extraction unit 22 as indicated by broken lines in FIG. 1, and the parameter may further include a positional relationship between the head portion and the trunk.

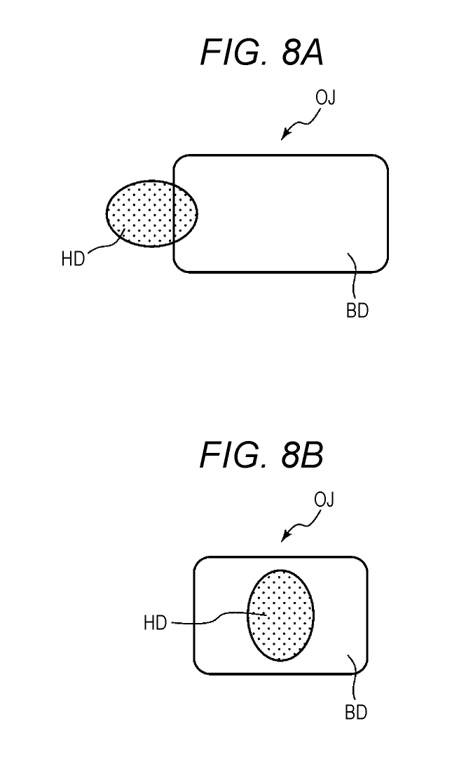

FIG. 8 is a diagram for description of a positional relationship between a head portion and a trunk in the sixth modification embodiment. FIG. 8A illustrates a state in which the monitored subject is lying, and FIG. 8B illustrates a state in which the monitored subject is squatting down and not lying. As illustrated in FIG. 8A, in the case where the longitudinal direction of a trunk BD coincides with the longitudinal direction of a head portion HD or where the head portion HD is positioned at one end of the trunk BD, it can be determined that lying is taken, and, as illustrated in FIG. 8B, in the case where the head portion HD is positioned at a center position of the trunk BD, it can be determined that squatting down is taken. A known image processing technique is used for extraction of the trunk BD. For example, the trunk BD is determined by the parameter calculation unit 231 via template matching using an outline shape of the trunk BD as a template that has been prepared in advance. It is noted that the template of the trunk BD may include an outline shape of a foot. In addition, for example, the trunk BD may be determined via, for example, moving object extraction using a background subtraction method. In the background subtraction method, a background image is determined and stored in advance, and a moving object is extracted as the trunk BD from a difference image between the acquired image and the background image.

In addition, in the embodiments described above, the image acquisition unit 1 may acquire plural images of the detection area at mutually different times, the head portion extraction unit 22 may extract a head portion from each of the plural images of the detection area acquired by the image acquisition unit 1, and the posture determination unit 23 may determine, as the parameter, a movement speed of the head portion on the basis of the plural head portions extracted by the head portion extraction unit 22, and may determine whether the prescribed posture is taken on the basis of the determined movement speed of the head portion. More specifically, the movement speed for determining whether fall-down/fall-off is taken is preset as a threshold value th3, and the posture determination unit 23 determines whether fall-down/fall-off is taken on the basis of whether or not the movement speed of the head portion is equal to or larger than the threshold value th3. It is highly possible that a relatively quick movement of the head portion indicates falling down/falling off. Accordingly, such a posture detection device D uses the movement speed of the head portion as the parameter, and thus can determine fall-down/fall-off serving as the prescribed posture of the monitored subject.

As described above, techniques of various embodiments are disclosed in the present description. Typical techniques among those techniques are summarized below.

A posture detection device according to an embodiment includes an image acquisition unit that acquires an image of a prescribed detection area, a head portion extraction unit that extracts a head portion from the image of the detection area acquired by the image acquisition unit, and a posture determination unit that determines a prescribed parameter for the head portion extracted by the head portion extraction unit and determines whether a prescribed posture is taken on a basis of the determined parameter.

In such a posture detection device, the image acquisition unit acquires an image of a detection area, the head extraction unit extracts a head portion (image region in which a head portion is captured in the image, or an image of a head portion) from the image of the detection area, and the posture determination unit determines a prescribed posture of a monitored subject (monitored person, watched person, or subject person) related to the head portion on the basis of a prescribed parameter for the head portion. Accordingly, in the posture detection device described above, the posture of the monitored subject, for example, fall-down or fall-off, can be determined more precisely with a simpler configuration of using a single image acquisition unit and by using a prescribed parameter for a head portion that is less likely to be blocked.

In another embodiment, in the posture detection device described above, the parameter is a size of the head portion in the image.

In the case where an image of the detection area is captured by viewing the detection area from above in a height direction of the monitored subject, the size of the head portion in the image is a size corresponding to the height of the head portion. Accordingly, the posture detection device described above can estimate, the height of the head portion can by using the size of the head portion as the parameter, and can determine the posture of the monitored subject such as the standing position, sitting position, and fall-down/fall-off on the basis of the estimated height of the head portion.

In another embodiment, in the posture detection device described above, the parameter is a height of the head portion.

Such a posture detection device uses the height of the head portion as the parameter, and thus can determine the posture of the monitored subject such as the standing position, sitting position, and fall-down/fall-off on the basis of the determined height of the head portion.

In another embodiment, in the posture detection devices described above, the parameter further includes a position of the head portion.

For example, even in the case where it is determined that fall-down/fall-off is taken from the size of the head portion or the height of the head portion, it is highly possible that the monitored subject has not fallen down/fallen off but is lying when the position of the head portion is on a bed, and conversely, it is highly possible that the monitored subject has fallen down/fallen off when the position of the head portion is on a floor. The posture detection devices described above use the position of the head portion for the determination of the posture in addition to the size of the head portion or the height of the head portion, and thus can determine the posture of the monitored subject even more precisely.

In another embodiment, in the posture detection devices described above, the parameter further includes an orientation of the head portion.

For example, even in the case where it is determined that fall-down/fall-off from the size of the head portion or the height of the head portion, it is highly possible that the monitored subject has not fallen down/fallen off but is squatting down when the orientation of the head portion, that is, the orientation of the face that can be determined from the orientation of the head portion directs straight to the front (in the horizontal direction), and, conversely, it is highly possible that the monitored subject has fallen down/fallen off when the orientation of the head portion, that is, the orientation of the face that can be determined from the orientation of the head portion directs sideways or upward. The posture detection device described above uses the orientation of the head portion (that is, the orientation of the face) for the determination of the posture in addition to the size of the head portion or the height of the head portion, and thus can determine the posture of the monitored subject even more precisely.

In another embodiment, in the posture detection devices described above, a trunk extraction unit that extracts, from the image of the detection area acquired by the image acquisition unit, a trunk corresponding to the head portion extracted by the head portion extraction unit is further included, and the parameter further includes a positional relationship between the head portion and the trunk.

With only the head portion extracted by the head portion extraction unit, a case in which it is difficult to determine the orientation of the head portion may occur. In this case, whether lying is taken can be determined by referring to the positional relationship between the head portion and the trunk (body). That is, it can be determined that lying is taken in the case where the head portion is positioned at one end of the trunk. The posture detection device further includes the trunk extraction unit, and, since the trunk (image region in which the trunk (body) is captured in the image, or an image of the trunk (body) is extracted from the image of the detection area by the trunk extraction unit and the positional relationship between the head portion and the trunk is used for the determination of the posture in addition to the size of the head portion or the height of the head portion, the posture of the monitored subject can be determined even more precisely.

In another embodiment, in the posture detection devices described above, the posture determination unit determines whether the prescribed posture is taken on a basis of whether or not the prescribed parameter for the head portion extracted by the head portion extraction unit is equal to or larger than a prescribed threshold value.

Such posture detection devices can easily determine whether the prescribed posture is taken by just determining whether or not the parameter is equal to or larger than the threshold value.

In another embodiment, in the posture detection devices described above, the threshold value is set on a basis of a height of a standing position.

The height of the sitting position varies depending on the height of the standing position, that is, the stature. Therefore, by setting the threshold value so as to be lower than the height of the sitting position on the basis of the height of the standing position (stature), the posture detection device described above becomes capable of determining whether the posture of the monitored subject is fallen down/fallen off.

In another embodiment, in the posture detection devices described above, the threshold value is set on a basis of a height of a sitting position.

In such posture detection devices, by setting the threshold value so as to be lower than the height of the sitting position on the basis of the height of the sitting position, the posture detection devices described above become capable of determining whether the posture of the monitored subject is fallen down/fallen off.

In another embodiment, the posture detection devices described above further include a first threshold value setting unit that sets the threshold value for each subject person.

Whereas a general-purpose posture detection device can be configured by setting the threshold value by performing statistical processing by using plural samples, it is more preferable that customization (optimization) is performed in accordance with the monitored subject. Since the posture detection devices described above further include the first threshold value setting unit and can set the threshold value in correspondence with the monitored subject, customization can be performed in accordance with the monitored subject (for each monitored person), and thus the posture of the monitored subject can be determined even more precisely.

In another embodiment, in the posture detection devices described above, the image acquisition unit acquires plural images of the detection area at mutually different times, and the posture detection devices further include a second threshold value setting unit that sets the threshold value on a basis of the plural images acquired by the image acquisition unit.

Since such posture detection devices set the threshold value by the second threshold value setting unit on the basis of plural images of the detection area at mutually different times, the threshold value can be set automatically for each subject person. In particular, even in the case where the posture of the standing position or walking is different from a healthy person as a result of, for example, a bent back, the threshold values can be set automatically in consideration of such a personal circumstance.

In another embodiment, the posture detection devices described above further include a threshold value correction unit that corrects the threshold value.

In the case of capturing an image of the detection area with a wide angle of view or via swing and tilt photographing, the size of the head portion in the image and the actual height of the head portion are not in a proportional relationship. The posture detection devices described above further include the threshold value correction unit that corrects the threshold value, and thus can correct the threshold value appropriately in accordance with an imaging condition and determine the posture of the monitored subject more precisely.

In another embodiment, in the described posture detection device, the threshold value is set to a different value for each of plural determination areas into which the detection area is divided.

Since the threshold value is set to a different value for each of the plural determination areas in such a posture detection device, it becomes possible to perform determination in consideration of the change in the relationship between the size and height of the head portion depending on the position in the image. In addition, according to this, it becomes possible to perform determination in consideration of a specific area in which a bed or the like is present.

In another embodiment, in the posture detection devices described above, the posture determination unit determines whether fall-down/fall-off serving as the prescribed posture is taken on the basis of whether the position of the head portion extracted by the head portion extraction unit is on a floor.

In the case where the position of the head portion is on the floor, it is highly possible that the posture of the monitored subject is fallen down/fallen off. The posture detection devices described above determine, by the posture determination unit, whether fall-down/fall-off serving as the prescribed posture is taken on the basis of whether the position of the head portion is on the floor, and thus can make determination of fall-down/fall-off more precisely.

In another embodiment, in the posture detection devices described above, the posture determination unit determines whether fall-down/fall-off serving as the prescribed posture is taken on the basis of whether the position of the head portion extracted by the head portion extraction unit is on a bed.

In the case where the position of the head portion is on the bed, it is highly possible that the posture of the monitored subject is not fallen down/fallen off but is lying on the bed. The posture detection devices described above determine, by the posture determination unit, whether fall-down/fall-off serving as the prescribed posture is taken on the basis of whether the position of the head portion is on the bed, and thus can make determination of fall-down/fall-off more precisely. In other words, lying on the bed can be determined.

In another embodiment, in the posture detection devices described above, the image acquisition unit acquires plural images of the detection area at mutually different times, the head portion extraction unit extracts a head portion from each of the plural images of the detection area acquired by the image acquisition unit, and the posture determination unit determines, as the parameter, a movement speed of the head portion on a basis of plural head portions extracted by the head portion extraction unit, and determines whether the prescribed posture is taken on a basis of the determined movement speed of the head portion.

It is highly possible that a relatively quick movement of the head portion indicates falling down/falling off. The posture detection devices use the movement speed of the head portion as the parameter, and thus can determine fall-down/fall-off serving as the prescribed posture of the monitored subject.

In another embodiment, in the posture detection devices described above, the image acquisition unit acquires plural images of the detection area at mutually different times, the head portion extraction unit extracts a head portion from each of the plural images of the detection area acquired by the image acquisition unit, the posture determination unit determines, for each of the plural images of the detection area acquired by the image acquisition unit, whether a prescribed posture is taken on a basis of a prescribed parameter for the head portion extracted by the head portion extraction unit, and the posture detection device further includes a final determination unit that makes final determination of whether the prescribed posture is taken on a basis of plural determination results of determination by the posture determination unit.

In such posture detection devices, the final determination unit makes final determination whether the prescribed posture is taken on the basis of plural determination results determined by the posture determination unit, and thus the posture of the monitored subject can be determined more precisely.

In another embodiment, in the posture detection devices described above, the image acquisition unit is a camera that captures an image of the detection area and is disposed on a ceiling.

In such posture detection devices, the camera serving as the image acquisition is disposed on the ceiling, and thus the monitored subject captured in the image of the detection area is less likely to be blocked by furniture or the like placed in the room, and the posture of the monitored subject can be determined more precisely.

A posture detection method according to another embodiment includes an image acquisition step of acquiring an image of a prescribed detection area, a head portion extraction step of extracting a head portion from the image of the detection area acquired in the image acquisition step, and a posture determination step of determining whether a prescribed posture is taken on a basis of a prescribed parameter for the head portion extracted in the head portion extraction step.

In such a posture detection method, an image of a detection area is acquired in an image acquisition step using an image acquisition unit, a head portion is extracted from the image of the detection area in the head portion extraction step, and a prescribed posture of a monitored subject related to the head portion is determined on the basis of a prescribed parameter for the head portion in the posture determination step. Accordingly, in the posture detection method described above, the posture of the monitored subject, for example, fall-down or fall-off, can be determined more precisely with a simpler configuration of using a single image acquisition unit and by using a prescribed parameter for a head portion.

This application is based on Japanese Patent Application No. 2015-44627 filed on Mar. 6, 2015, and the content thereof is included in the present application.

In order to express the present invention, the present invention has been described above appropriately and sufficiently through embodiments with reference to drawings, and it should be recognized that one skilled in the art can easily modify and/or improve the embodiments described above. Therefore, it is interpreted that modified embodiments and improved embodiments implemented by one skilled in the art are included in the scope of the claims as long as the modification embodiments and the improved embodiments do not deviate from the scope of right of the claims

### INDUSTRIAL APPLICABILITY

According to the present invention, a posture detection device that detects the posture of a monitored subject and a posture detection method can be provided.

1. A posture detection device comprising:
an image acquisitor that acquires an image of a prescribed detection area;
a head portion extractor that extracts a head portion from the image of the detection area acquired by the image acquisitor; and
a posture determiner that determines a prescribed parameter for the head portion extracted by the head portion extractor and determines whether a prescribed posture is taken on a basis of the determined parameter.

2. The posture detection device according to claim 1, wherein the parameter is a size of the head portion in the image.

3. The posture detection device according to claim 1, wherein the parameter is a height of the head portion.

4. The posture detection device according to claim 2, wherein the parameter further includes a position of the head portion.

5. The posture detection device according to claim 2, wherein the parameter further includes an orientation of the head portion.

6. The posture detection device according to claim 2, further comprising
a trunk extractor that extracts, from the image of the detection area acquired by the image acquisitor, a trunk corresponding to the head portion extracted by the head portion extractor,
wherein the parameter further includes a positional relationship between the head portion and the trunk.

7. The posture detection device according to claim 1, wherein the posture determiner determines whether the prescribed posture is taken on a basis of whether or not the prescribed parameter for the head portion extracted by the head portion extractor is equal to or larger than a prescribed threshold value.

8. The posture detection device according to claim 7, wherein the threshold value is set on a basis of a height of a standing position.

9. The posture detection device according to claim 7, wherein the threshold value is set on a basis of a height of a sitting position.

10. The posture detection device according to claim 7, further comprising a first threshold value setter that sets the threshold value for each subject person.

11. The posture detection device according to claim 7, wherein the image acquisitor acquires plural images of the detection area at mutually different times, the posture detection device further comprising a second threshold value setter that sets the threshold value on a basis of the plural images acquired by the image acquisitor.

12. The posture detection device according to claim 7, further comprising a threshold value corrector that corrects the threshold value.

13. The posture detection device according to claim 12, wherein the threshold value is set to a different value for each of the plural determination areas into which the detection area is divided.

14. The posture detection device according to claim 4, wherein the posture determiner determines whether fall-down/fall-off serving as the prescribed posture is taken on a basis of whether the position of the head portion extracted by the head portion extractor is on a floor.

15. The posture detection device according to claim 4, wherein the posture determiner determines whether fall-down/fall-off serving as the prescribed posture is taken on a basis of whether the position of the head portion extracted by the head portion extractor is on a bed.

16. The posture detection device according to claim 1, wherein
the image acquisitor acquires plural images of the detection area at mutually different times,
the head portion extractor extracts a head portion from each of the plural images of the detection area acquired by the image acquisitor, and
the posture determiner determines, as the parameter, a movement speed of the head portion on a basis of plural head portions extracted by the head portion extractor, and determines whether the prescribed posture is taken on a basis of the determined movement speed of the head portion.

17. The posture detection device according to claim 1, wherein
the image acquisitor acquires plural images of the detection area at mutually different times,
the head portion extractor extracts a head portion from each of the plural images of the detection area acquired by the image acquisitor,
the posture determination unit determines, for each of the plural images of the detection area acquired by the image acquisitor, whether a prescribed posture is taken on a basis of a prescribed parameter for the head portion extracted by the head portion extraction unit, and
the posture detection device further includes a final determiner that makes final determination of whether the prescribed posture is taken on a basis of plural determination results of determination by the posture determiner.

18. The posture detection device according to claim 1, wherein the image acquisitor is a camera that captures an image of the detection area and is disposed on a ceiling.

19. A posture detection method comprising:
an image acquisition step of acquiring an image of a prescribed detection area;
a head portion extraction step of extracting a head portion from the image of the detection area acquired in the image acquisition step; and
a posture determination step of determining whether a prescribed posture is taken on a basis of a prescribed parameter for the head portion extracted in the head portion extraction step.

20. The posture detection device according to claim 2, wherein the posture determiner determines whether the prescribed posture is taken on a basis of whether or not the prescribed parameter for the head portion extracted by the head portion extractor is equal to or larger than a prescribed threshold value.
